(19) United States
(12) Patent Application Publication
(10) Pub. No.: US 2014/0031908 A1
(43) Pub. Date: Jan. 30, 2014

(54) SYSTEM AND METHOD FOR ENHANCING LARGE DIAMETER NERVE FIBER STIMULATION USING SEQUENTIAL ACTIVATION OF ELECTRODES

(71) Applicant: BOSTON SCIENTIFIC NEUROMODULATION CORPORATION, Valencia, CA (US)

(72) Inventor: Dongchul Lee, Agua Dulce, CA (US)

(73) Assignee: BOSTON SCIENTIFIC NEUROMODULATION CORPORATION, Valencia, CA (US)

(21) Appl. No.: 13/945,221

(22) Filed: Jul. 18, 2013

Related U.S. Application Data
(60) Provisional application No. 61/676,824, filed on Jul. 27, 2012.

(51) Int. Cl. A61N 1/05

(52) U.S. Cl. CPC A61N 1/0551
USPC 607/117; 607/116; 607/118

(57) ABSTRACT

A system and method of providing therapy to a patient with electrodes extending along the longitudinal axes of large and small diameter nerve fibers. Electrical energy is delivered from a first electrode at an initial axial location, thereby evoking initial action potentials in the large and small fibers. The initial action potentials are allowed to be conducted along the large and small fibers in a first axial direction. Electrical energy is delivered from a second electrode at a second axial location at an instant in time when the initial action potential conducted along the large fiber has passed the second axial location and the initial action potential conducted along the small fiber is at the second axial location, thereby allowing a subsequent action potential to be evoked in the large fiber while preventing a subsequent action potential from being evoked in the small fiber.

### RELATED APPLICATION DATA

The present application claims the benefit under 35 U.S.C. §119 to U.S. provisional patent application Ser. No. 61/676,824, filed Jul. 27, 2012. The foregoing application is hereby incorporated by reference into the present application in its entirety.

### FIELD OF THE INVENTION

The present inventions relate to tissue stimulation systems, and more particularly, to systems and methods for therapeutically stimulating nerve fibers.

### BACKGROUND OF THE INVENTION

Implantable neurostimulation systems have proven therapeutic in a wide variety of diseases and disorders. Pacemakers and Implantable Cardiac Defibrillators (ICDs) have proven highly effective in the treatment of a number of cardiac conditions (e.g., arrhythmias). Spinal Cord Stimulation (SCS) systems have long been accepted as a therapeutic modality for the treatment of chronic pain syndromes, and the application of spinal stimulation has begun to expand to additional applications, such as angina pectoris and incontinence. Deep Brain Stimulation (DBS) has also been applied therapeutically for well over a decade for the treatment of refractory Parkinson's Disease, and DBS has also recently been applied in additional areas, such as essential tremor and epilepsy. Further, in recent investigations, Peripheral Nerve Stimulation (PNS) systems have demonstrated efficacy in the treatment of chronic pain syndromes and incontinence, and a number of additional applications are currently under investigation. Furthermore, Functional Electrical Stimulation (FES) systems such as the Freehand system by NeuroControl (Cleveland, Ohio) have been applied to restore some functionality to paralyzed extremities in spinal cord injury patients.

Each of these implantable neurostimulation systems typically includes one or more electrode carrying stimulation leads, which are implanted at the desired stimulation site, and a neurostimulation device implanted remotely from the stimulation site, but coupled either directly to the neurostimulation leads or indirectly to the neurostimulation leads via a lead extension. Thus, electrical pulses can be delivered from the neurostimulation device to the electrodes to activate a volume of tissue in accordance with a set of stimulation parameters and provide the desired efficacious therapy to the patient. In particular, electrical energy conveyed between at least one cathodic electrode and at least one anodic electrode creates an electrical field, which when strong enough, depolarizes (or “stimulates”) the neurons beyond a threshold level, thereby inducing the firing of action potentials (APs) that propagate along the neural fibers. A typical stimulation parameter set may include the electrodes that are sourcing (anodes) or returning (cathodes) the modulating current at any given time, as well as the amplitude, duration, and rate of the stimulation pulses.

The neurostimulation system may further comprise a handheld patient programmer to remotely instruct the neurostimulation device to generate electrical stimulation pulses in accordance with selected stimulation parameters. The handheld programmer in the form of a remote control (RC) may, itself, be programmed by a clinician, for example, by using a clinician's programmer (CP), which typically includes a general purpose computer, such as a laptop, with a programming software package installed thereon.

To better understand the effect of stimulation pulses on nerve tissue, reference to FIG. 1 will now be made. As there shown, a typical neuron 1 that can be found in the white matter of the spinal cord or brain includes an axon 2 containing ionic fluid (and primarily potassium and sodium ions) 3, a myelin sheath 4, which is formed of a fatty tissue layer, coating the axon 2, and a series of regularly spaced gaps 5 (referred to as “Nodes of Ranvier”), which are typically about 1 micrometer in length and expose a membrane 6 of the axon 2 to extracellular ionic fluid 7.

When the neuron 1 is stimulated, e.g., via an electrical pulse, an action potential (i.e., a sharp electrochemical response) is induced within the neuron 1. As a result, a transmembrane voltage potential (i.e., a voltage potential that exists across the membrane 6 of the axon 3) changes, thereby conducting a neural impulse along the axon neuron 1 as sodium and potassium ions flow in and out of the axon 3 via the ion channels in the membrane 6. Because ion flow can only occur at the nodes 5 where the membrane 6 of the axon 3 is exposed to the extracellular ionic fluid 3, the neural impulse will actually jump along the axon 3 from one node to the next node. In this manner, the myelin sheath 4 serves to velocity the neural impulse by insulating the electrical current and making it possible for the impulse to jump from node to node along the axon 3, which is faster and more energetically favorable than continuous conduction along the axon 3. Immediately after the neural impulse is conducted, the respective node 5 enters a refractory period during which an action potential cannot be induced at the node 5. Further details discussing the electro-chemical mechanisms involved with propagating an AP along a neuron are disclosed in U.S. patent Ser. No. 11/752,895, entitled “Short Duration Pre-Pulsing to Reduce Stimulation-Evoked Side-Effects,” which is expressly incorporated herein by reference.

Typically, the therapeutic effect for any given neurostimulation application may be optimized by adjusting the stimulation parameters. Often, these therapeutic effects are correlated to the diameter of the nerve fibers that innervate the volume of tissue to be modulated. For example, in SCS, activation (i.e., recruitment) of large diameter sensory fibers is believed to reduce/block transmission of smaller diameter pain fibers via interneuronal interaction in the dorsal horn of the spinal cord. Activation of large sensory fibers also typically creates a sensation known as paresthesia that can be characterized as an alternative sensation that replaces the pain signals sensed by the patient.

In electrical stimulation, recruitment order is determined by the size of the nerve fiber. That is, because larger nerve fibers have lower stimulation thresholds than smaller nerve fibers, the larger nerve fibers will normally be stimulated before smaller nerve fibers when located the same distance from the active electrode or electrodes. For maximum therapeutic effect, a certain amount of amplitude is required. Usually, the maximum amplitude is determined by a discomfort sensation of stimulation, typically pain, uncomfortable paresthesia, or an uncomfortable sensation in a joint. It is thought that these discomfort sensations may be associated with the stimulation of small diameter nerve fibers. It would therefore be desirable to reduce the stimulation of small diameter nerve fibers with respect to the stimulation of large diameter nerve fibers in order to maximize the therapeutic outcome of electrical stimulation therapy.

### SUMMARY OF THE INVENTION

In accordance with one aspect of the present inventions, a neurostimulation system comprises a plurality of electrical terminals configured for being respectively coupled to a plurality of electrodes, analog output circuitry configured for generating a train of stimulation pulses, and control circuitry configured for instructing the analog output circuitry to sequentially deliver a first one of the stimulation pulses to a first one of the electrical terminals, and a second one of the stimulation pulses to a second one of the electrical terminals. In an optional embodiment, the neurostimulation system further comprises a user interface configured for receiving an input from the user defining a pulse rate between the first and second stimulation pulses. The electrodes may be carried by a neurostimulation lead in an electrode column. The pulse rate may be in the range

$$\frac{20 mm \text{/} ms}{x} \leq r \leq \frac{120 mm \text{/} ms}{x} ,$$

where r is the pulse rate in pulses per millisecond, and x is the electrode spacing in millimeters. The neurostimulation system may further comprises a housing containing the plurality of electrical terminals, the analog output circuitry, and the control circuitry.

In accordance with a second aspect of the present inventions, a method of providing therapy to a patient with a plurality of electrodes extending along the longitudinal axes of a relatively large diameter nerve fiber and a relatively small diameter nerve fiber (e.g., spinal cord nerve fibers, such as dorsal column nerve fibers) is provided. The series of electrodes may be carried by a neurostimulation lead and may be implanted within the patient. The method comprises delivering electrical energy from a first one of the electrodes at an initial axial location, thereby evoking an initial action potential in the large diameter nerve fiber, and an initial action potential in the small diameter nerve fiber, and allowing the respective initial action potentials to be conducted along the large diameter nerve fiber and the small diameter nerve fiber in a first axial direction towards a second axial location. Preferably, the initial action potential in the large diameter nerve fiber has a conduction velocity greater than a conduction velocity of the initial action potential in the small diameter nerve fiber.

The method further comprises delivering electrical energy from a second one of the electrodes at the second axial location at an instant in time when the initial action potential conducted along the large diameter nerve fiber has passed the second axial location and the initial action potential conducted along the small diameter nerve fiber is at the second axial location, thereby allowing a subsequent action potential to be evoked in the large diameter nerve fiber while preventing a subsequent action potential from being evoked in the small diameter nerve fiber. For example, the small diameter nerve fiber may be undergoing a refractory period at the second axial location when the electrical energy is delivered from the second electrode at the second axial location. The first and second electrodes are immediately adjacent each other. Preferably, therapy to the patient is provided using a train of pulses, in which case, the delivery of the electrical energy from the first and second electrodes respectively comprises sequentially delivering first and second pulses of the pulse train from the first and second electrodes. Preferably, no electrical energy is delivered from the first electrode when the electrical energy is delivered from the second electrode.

An optional method further comprises allowing the respective initial action potentials to be conducted along the large diameter nerve fiber and the small diameter nerve fiber in the first axial direction from the second axial location towards a third axial location, and delivering electrical energy from a third one of the electrodes at the third axial location at an instant in time when the initial action potential conducted along the large diameter nerve fiber has passed the third axial location and the initial action potential conducted along the small diameter nerve fiber is at the third axial location, thereby allowing another subsequent action potential to be evoked in the large diameter nerve fiber while preventing a subsequent action potential from being evoked in the small diameter nerve fiber.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a plan view of a neural axon;

FIG. 2 is a plan view of one embodiment of a spinal cord stimulation (SCS) system arranged in accordance with the present inventions;

FIG. 3 is a plan view of an implantable pulse generator (IPG) and stimulation leads used in the SCS system of FIG. 2;

FIG. 4 is a plan view of the SCS system of FIG. 3 in use with a patient;

FIG. 5 is a timing diagram showing how action potentials are respectively propagated over time along a large diameter nerve fiber and small diameter nerve fiber as a function of electrode spacing;

FIG. 6 is a timing diagram showing how additional action potentials may be evoked in the large diameter nerve fiber without generating additional action potentials in the small diameter nerve fiber by applying a series of stimulation pulses one electrode at a time at the conduction velocity of the small diameter nerve fiber;

FIG. 7 is a block diagram of the internal components of the IPG of FIG. 3;

FIG. 8 is front view of a remote control (RC) used in the neurostimulation system of FIG. 2; and

FIG. 9 is a block diagram of the internal components of the RC of FIG. 8.

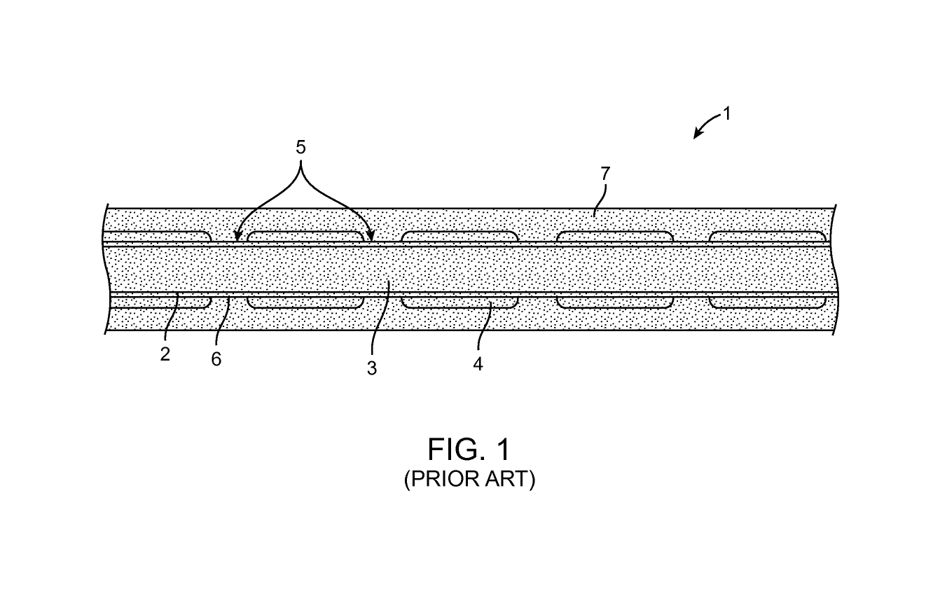

### DETAILED DESCRIPTION OF THE EMBODIMENTS

The description that follows relates to a spinal cord stimulation (SCS) system. However, it is to be understood that while the invention lends itself well to applications in SCS, the invention, in its broadest aspects, may not be so limited. Rather, the invention may be used with any type of implantable electrical circuitry used to stimulate tissue. For example, the present invention may be used as part of a multi-lead system such as a pacemaker, a defibrillator, a cochlear stimulator, a retinal stimulator, a stimulator configured to produce coordinated limb movement, a cortical stimulator, a deep brain stimulator, peripheral nerve stimulator, microstimulator, or in any other neural stimulator configured to treat urinary incontinence, sleep apnea, shoulder sublaxation, headache, etc.

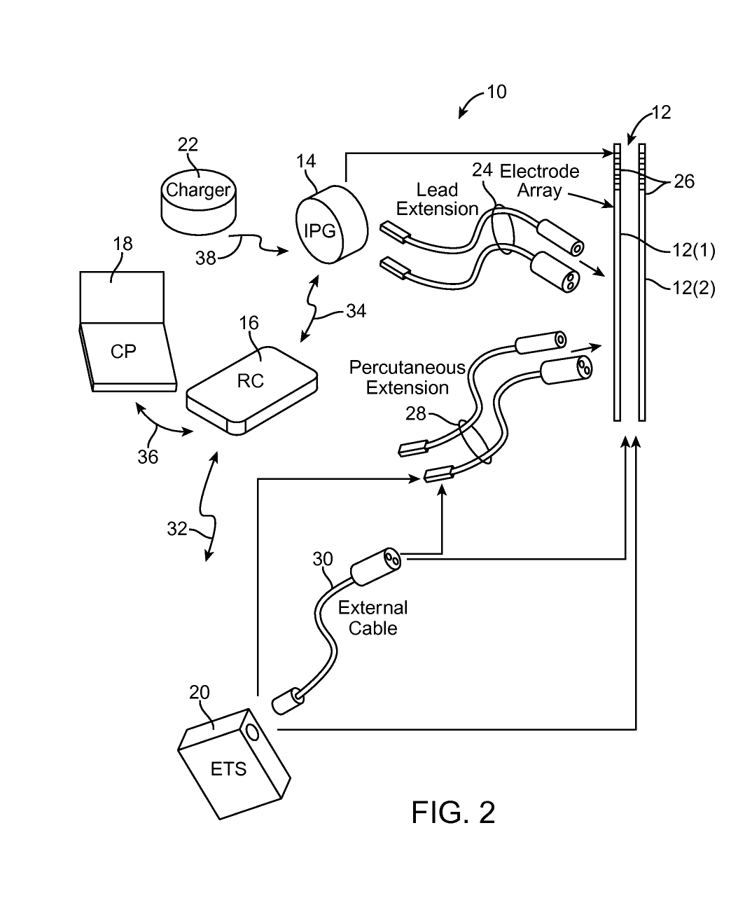

Turning first to FIG. 2, an exemplary SCS system 10 generally comprises a plurality of percutaneous neurostimulation leads 12 (in this case, two percutaneous leads 12(1) and 12(2)), an implantable pulse generator (IPG) 14, an external remote control (RC) 16, a Clinician's Programmer (CP) 18, an External Trial Stimulator (ETS) 20, and an external charger 22.

The IPG 14 is physically connected via one or more percutaneous lead extensions 24 to the neurostimulation leads 12, which carry a plurality of electrodes 26 arranged in an array. In the illustrated embodiment, the neurostimulation leads 12 are percutaneous leads, and to this end, the electrodes 26 are arranged in-line along the neurostimulation leads 12. Alternatively, a surgical paddle lead can be used in place of or in addition to the percutaneous leads. As will be described in further detail below, the IPG 14 includes pulse generation circuitry that delivers electrical stimulation energy in the form of a pulsed electrical waveform (i.e., a temporal series of electrical pulses) to the electrode array 26 in accordance with a set of stimulation parameters.

The ETS 20 may also be physically connected via the percutaneous lead extensions 28 and external cable 30 to the neurostimulation leads 12. The ETS 20, which has similar pulse generation circuitry as the IPG 14, also delivers electrical stimulation energy in the form of a pulse electrical waveform to the electrode array 26 accordance with a set of stimulation parameters. The major difference between the ETS 20 and the IPG 14 is that the ETS 20 is a non-implantable device that is used on a trial basis after the neurostimulation leads 12 have been implanted and prior to implantation of the IPG 14, to test the responsiveness of the stimulation that is to be provided. Thus, any functions described herein with respect to the IPG 14 can likewise be performed with respect to the ETS 20.

The RC 16 may be used to telemetrically control the ETS 20 via a bi-directional RF communications link 32. Once the IPG 14 and neurostimulation leads 12 are implanted, the RC 16 may be used to telemetrically control the IPG 14 via a bi-directional RF communications link 34. Such control allows the IPG 14 to be turned on or off and to be programmed with different stimulation programs after implantation. Once the IPG 14 has been programmed, and its power source has been charged or otherwise replenished, the IPG 14 may function as programmed without the RC 16 being present.

The CP 18 provides clinician detailed stimulation parameters for programming the IPG 14 and ETS 20 in the operating room and in follow-up sessions. The CP 18 may perform this function by indirectly communicating with the IPG 14 or ETS 20, through the RC 16, via an IR communications link 36. Alternatively, the CP 18 may directly communicate with the IPG 14 or ETS 20 via an RF communications link (not shown).

The external charger 22 is a portable device used to transcutaneously charge the IPG 14 via an inductive link 38. Once the IPG 14 has been programmed, and its power source has been charged by the external charger 22 or otherwise replenished, the IPG 14 may function as programmed without the RC 16 or CP 18 being present.

For purposes of brevity, the details of the CP 18, ETS 20, and external charger 22 will not be described herein. Details of exemplary embodiments of these devices are disclosed in U.S. Pat. No. 6,895,280, which has been previously incorporated herein by reference.

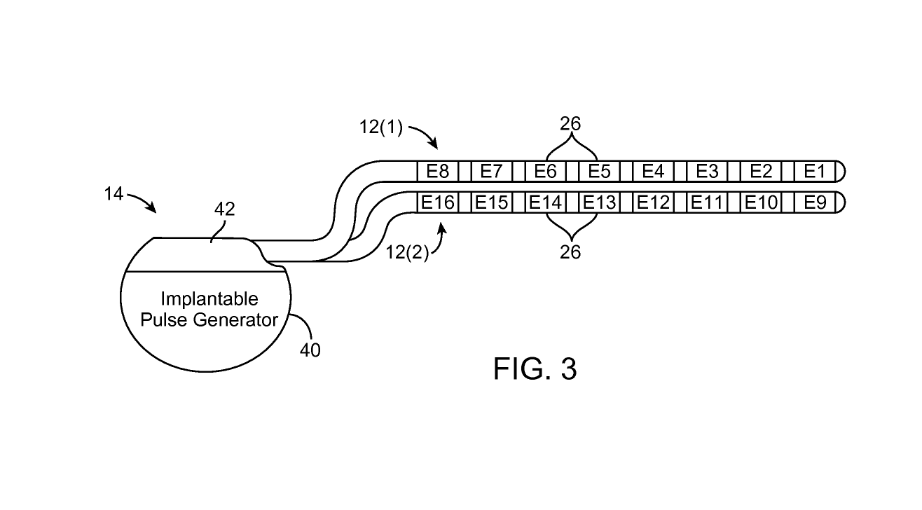

Referring now to FIG. 3, the external features of the neurostimulation leads 12 and the IPG 14 will be briefly described. Each of the neurostimulation leads 12 has eight electrodes 26 (respectively labeled E1-E8 for the lead 12(1) and E9-E16 for the lead 12(2)). The actual number and shape of leads and electrodes will, of course, vary according to the intended application. Further details describing the construction and method of manufacturing percutaneous stimulation leads are disclosed in U.S. patent application Ser. No. 11/689,918, entitled “Lead Assembly and Method of Making Same,” and U.S. patent application Ser. No. 11/565,547, entitled “Cylindrical Multi-Contact Electrode Lead for Neural Stimulation and Method of Making Same,” the disclosures of which are expressly incorporated herein by reference.

The IPG 14 comprises an outer case 40 for housing the electronic and other components (described in further detail below). The outer case 40 is composed of an electrically conductive, biocompatible material, such as titanium, and forms a hermetically sealed compartment wherein the internal electronics are protected from the body tissue and fluids. In some cases, the outer case 40 may serve as an electrode. The IPG 14 further comprises a connector 42 to which the proximal ends of the neurostimulation leads 12 mate in a manner that electrically couples the electrodes 26 to the internal electronics (described in further detail below) within the outer case 40. To this end, the connector 42 includes two ports (not shown) for receiving the proximal ends of the three percutaneous leads 12. In the case where the lead extensions 24 are used, the ports may instead receive the proximal ends of such lead extensions 24.

As will be described in further detail below, the IPG 14 includes pulse generation circuitry that provides electrical stimulation energy to the electrodes 26 in accordance with a set of parameters. Such parameters may comprise electrode combinations, which define the electrodes that are activated as anodes (positive), cathodes (negative), and turned off (zero), and electrical pulse parameters, which define the pulse amplitude (measured in milliamps or volts depending on whether the IPG 14 supplies constant current or constant voltage to the electrodes), pulse duration (measured in microseconds), pulse rate (measured in pulses per second), and pulse shape.

With respect to the pulse patterns provided during operation of the SCS system 10, electrodes that are selected to transmit or receive electrical energy are referred to herein as “activated,” while electrodes that are not selected to transmit or receive electrical energy are referred to herein as “non-activated.” Electrical energy delivery will occur between two (or more) electrodes, one of which may be the IPG case 40, so that the electrical current has a path from the energy source contained within the IPG case 40 to the tissue and a sink path from the tissue to the energy source contained within the case. Electrical energy may be transmitted to the tissue in a monopolar or multipolar (e.g., bipolar, tripolar, etc.) fashion.

Monopolar delivery occurs when a selected one or more of the lead electrodes 26 is activated along with the case 40 of the IPG 14, so that electrical energy is transmitted between the selected electrode 26 and case 40. Monopolar delivery may also occur when one or more of the lead electrodes 26 are activated along with a large group of lead electrodes located remotely from the one or more lead electrodes 26 so as to create a monopolar effect; that is, electrical energy is conveyed from the one or more lead electrodes 26 in a relatively isotropic manner. Bipolar delivery occurs when two of the lead electrodes 26 are activated as anode and cathode, so that electrical energy is transmitted between the selected electrodes 26. Tripolar delivery occurs when three of the lead electrodes 26 are activated, two as anodes and the remaining one as a cathode, or two as cathodes and the remaining one as an anode.

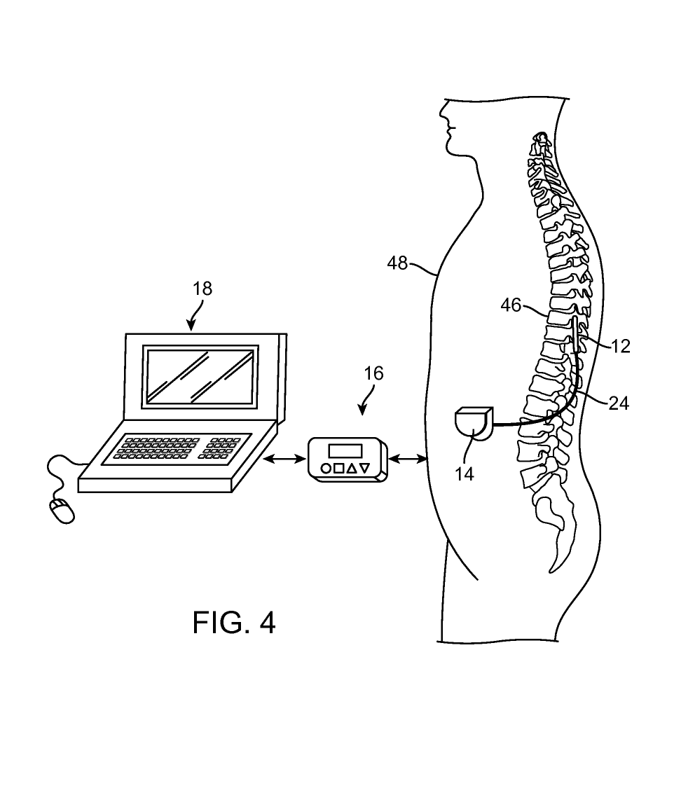

Referring to FIG. 4, the neurostimulation leads 12 are implanted within the spinal column 46 of a patient 48. The preferred placement of the neurostimulation leads 12 is adjacent, i.e., resting near, or upon the dura, adjacent to the spinal cord area to be stimulated. Due to the lack of space near the location where the neurostimulation leads 12 exit the spinal column 46, the IPG 14 is generally implanted in a surgically-made pocket either in the abdomen or above the buttocks. The IPG 14 may, of course, also be implanted in other locations of the patient's body. The lead extensions 24 facilitate locating the IPG 14 away from the exit point of the neurostimulation leads 12. As there shown, the CP 18 communicates with the IPG 14 via the RC 16. While the neurostimulation leads 12 are illustrated as being implanted near the spinal cord area of a patient, the neurostimulation leads 12 may be implanted anywhere in the patient's body, including a peripheral region, such as a limb, or the brain. After implantation, the IPG 14 is used to provide the therapeutic stimulation under control of the patient.

More significant to the present inventions, the SCS system 10 may be operated in a manner that delivers high intensity stimulation energy to relatively large diameter neural axons, thereby providing therapy to a patient, while preventing over-stimulation of relatively small diameter neural axons, thereby minimizing or preventing adverse side-effects. This technique takes advantage of the natural phenomenon that small diameter nerve fibers generally have slower conduction velocitys, and thus a slower action potential propagation, than do large diameter nerve fibers.

In particular, this technique applies a series of stimulation pulses one electrode at a time along one or both of the neurostimulation leads 12 extending along the longitudinal axes of nerve fibers (i.e., substantially parallel to the longitudinal axes of the nerve fibers), which include relatively large diameter nerve fibers that are desired to be stimulated, and relatively small diameter nerve fibers that are desired to not be stimulated. Stimulation energy is initially applied to the nerve fibers at one end of the neurostimulation lead 12, and then successively applied to the nerve fibers along the neurostimulation lead 12 or another neurostimulation lead 12 at the velocity of the action potential propagation of the small diameter nerve fibers.

Because an action potential cannot again be evoked in a nerve fiber at a node of Ranvier that is presently in a refractory period caused by existence of a previous action potential, stimulation energy subsequently applied to this node of Ranvier will not evoke another action potential. Thus, if stimulation energy is serially applied along the neurostimulation lead 12 at the conduction velocity of the small diameter nerve fibers, only one initial action potential will be evoked in each of the small diameter nerve fibers. However, the large diameter nerve fibers will have recovered from the refractory period in time, such that the serially applied stimulation energy will evoke multiple action potentials in each of the large diameter nerve fibers. This technique may thus minimize activation of small diameter nerve fibers, while maximizing the activation of large diameter nerve fibers. That is, the firing rate of the large diameter nerve fibers is increased without evoking additional action potentials on the small diameter nerve fibers.

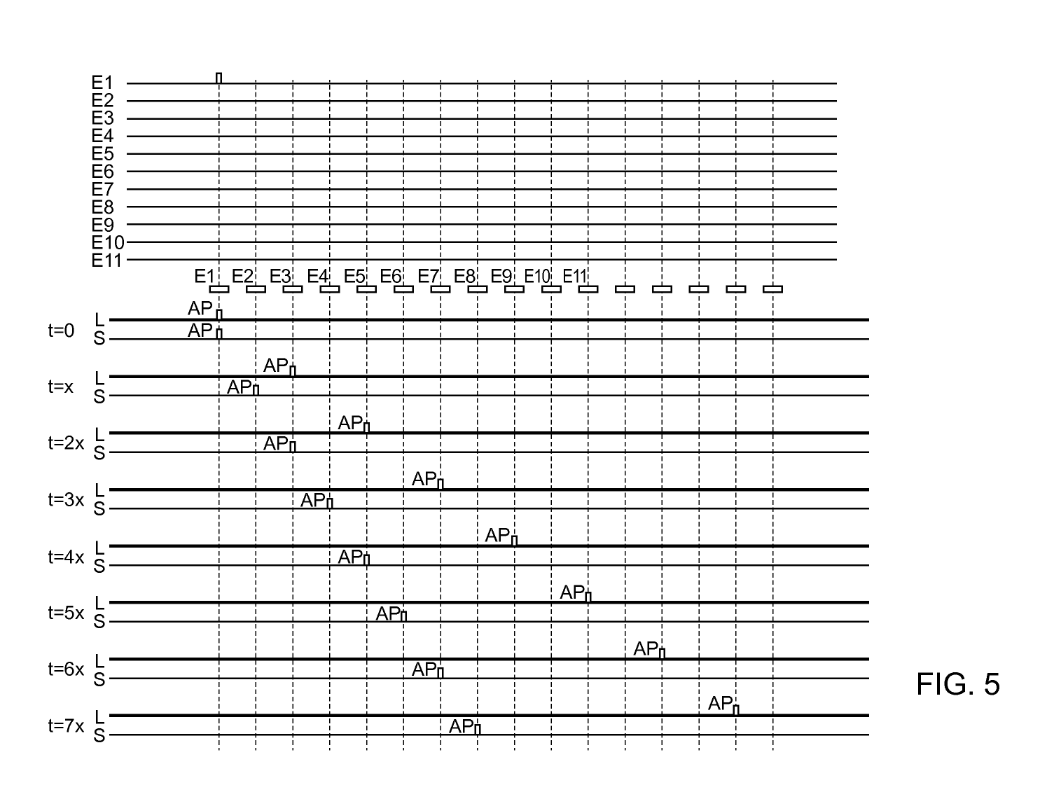

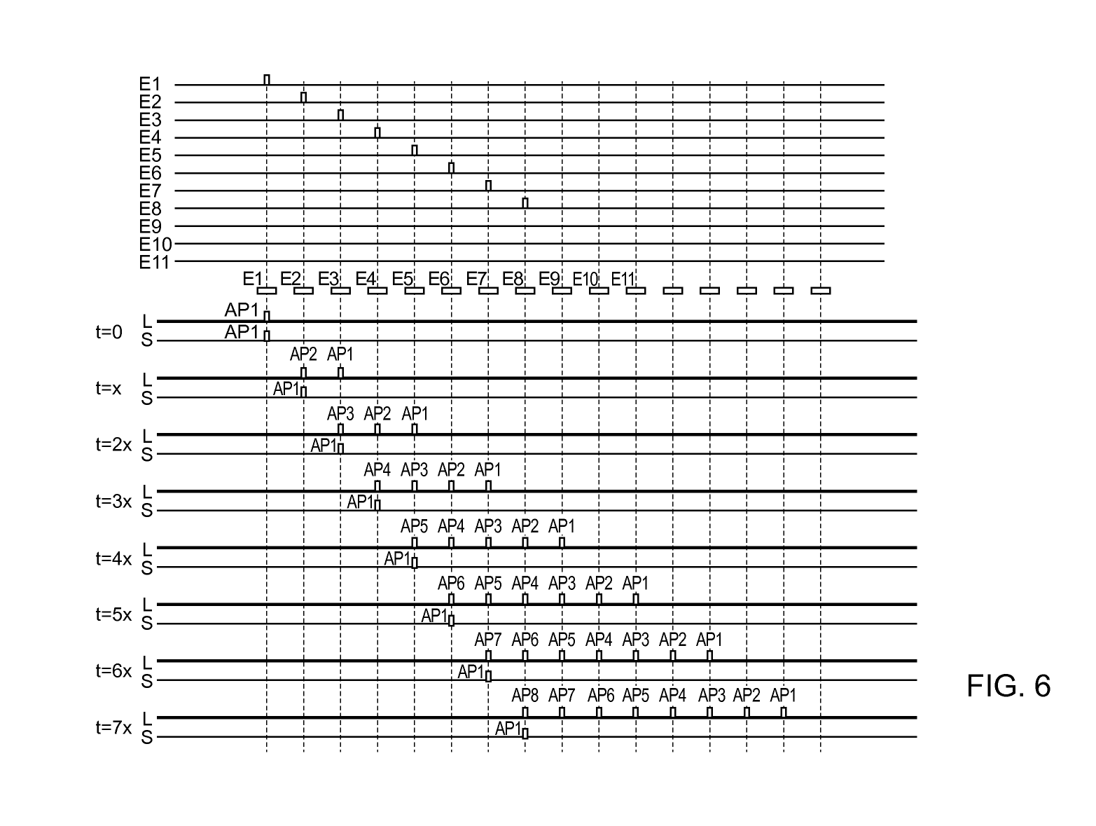

Referring now to FIGS. 5 and 6, one example of a technique for maximizing the activation of large diameter nerve fibers, while minimizing the activation of small diameter nerve fibers will be described.

FIG. 5 shows how action potentials are respectively propagated over time along a large diameter nerve fiber L and small diameter nerve fiber S as a function of electrode spacing. As is typical, the conduction velocity of the large diameter nerve fiber L is faster than, and in this case twice as fast as, the conduction velocity of the small diameter nerve fiber S. It is assumed that the time to take an action potential to propagate between successive electrodes for the small diameter nerve fiber S is the electrode spacing x, and the action potentials AP in the nerve fibers are initially evoked by an electrical pulse delivered by electrode E1 at t=0.

At t=x, the action potential AP in the large diameter nerve fiber L is at an axial location under electrode E3 and the action potential AP in the small diameter nerve fiber S is at an axial location under electrode E2; at t=2x, the action potential AP in the large diameter nerve fiber L is at an axial location under electrode E5 and the action potential AP in the small diameter nerve fiber S is at an axial location under electrode E3; at t=3x, the action potential AP in the large diameter nerve fiber L is at an axial location under electrode E7 and the action potential AP in the small diameter nerve fiber S is at an axial location under electrode E4, and so forth. Although FIG. 5 illustrates the unidirectional conveyance of action potentials along the nerve fibers in a single axial direction, in the typical case, action potentials are bi-directionally conveyed along the nerve fibers in opposite axial directions.

FIG. 6 shows how additional action potentials may be evoked in the large diameter nerve fiber L without generating additional action potentials in the small diameter nerve fiber S by applying a series of stimulation pulses one electrode at a time at the conduction velocity of the small diameter nerve fiber S. The rate of the series of stimulation pulses may be adjusted to match the conduction velocity of the small diameter nerve fiber by dividing the conduction velocity by the electrode spacing. The conduction velocity of a human nerve fiber is typically in the range of 20 mm/ms to 120 mm/ms, and thus, for a typical small diameter nerve fiber, the pulse rate may be adjusted in the range of range

$$\frac{20 mm \text{/} ms}{x} \leq r \leq \frac{120 mm \text{/} ms}{x} ,$$

where r is the pulse rate in pulses per millisecond, and x is the electrode spacing in millimeters.

Therefore, any discomfort from stimulating small diameter nerve fibers would not increase, while the total number of action potentials in the large diameter nerve fibers will increase, thereby maximizing therapy. In particular, at t=0, an electrical pulse is initially delivered by electrode E1 at an initial axial location adjacent electrode E1, thereby evoking an initial action potential AP1 in the large diameter nerve fiber L, and an initial action potential AP1 in the small diameter nerve fiber S. The initial action potentials AP1 are then conducted along the nerve fibers in a first axial direction towards the remaining electrodes E2-E15.

At t=x, the initial action potential AP1 in the large diameter nerve fiber L is at an axial location adjacent electrode E3, and the initial action potential AP1 in the small diameter nerve fiber S is at an axial location adjacent electrode E2. At t=x, an electrical pulse is delivered by electrode E2 at the axial location adjacent electrode E2. Because the initial action potential AP1 conducted along the large diameter nerve fiber L has already passed electrode E2 at t=x, the large diameter nerve fiber L has thus been released from the refractory period at the axial location adjacent electrode E2. The electrical pulse delivered by electrode E2 will therefore evoke another action potential AP2 in the large diameter nerve fiber L at this axial location. However, because, at t=x, the initial action potential AP1 conducted along the small diameter nerve fiber S at electrode E2, the small diameter nerve fiber S will be in the refractory period at the axial location adjacent electrode E2. The electrical pulse delivered by electrode E2 will, therefore, not evoke another action potential in the small diameter nerve fiber S at this axial location.

At t=2x, the action potentials AP1 and AP2 in the large diameter nerve fiber L are respectively at axial locations adjacent electrodes E4 and E5, and the initial action potential AP1 in the small diameter nerve fiber S is at an axial location adjacent electrode E3. At t=2x, an electrical pulse is delivered by electrode E3 at the axial location adjacent electrode E3. Because, at t=2x, the action potentials AP1 and AP2 conducted along the large diameter nerve fiber L have already passed electrode E3, the large diameter nerve fiber L has thus been released from the refractory period at the axial location adjacent electrode E3. The electrical pulse delivered by electrode E3 will therefore evoke another action potential A3 in the large diameter nerve fiber L at this axial location. However, because the initial action potential AP1 conducted along the small diameter nerve fiber S is at electrode E3 at t=2x, the small diameter nerve fiber S will be in the refractory period at the axial location adjacent electrode E3. The electrical pulse delivered by electrode E3 will, therefore, not evoke another action potential in the small diameter nerve fiber S at this axial location.

At t=3x, the action potentials AP1-AP3 in the large diameter nerve fiber L are respectively at axial locations adjacent electrodes E5-E7, and the initial action potential AP1 in the small diameter nerve fiber S is at an axial location adjacent electrode E4. At t=3x, an electrical pulse is delivered by electrode E4 at the axial location adjacent electrode E4. Because, at t=3x, the action potentials AP1-AP3 conducted along the large diameter nerve fiber L have already passed electrode E4, the large diameter nerve fiber L has thus been released from the refractory period at the axial location adjacent electrode E4. The electrical pulse delivered by electrode E4 will, therefore, evoke another action potential A4 in the large diameter nerve fiber L at this axial location. However, because, at =3x, the initial action potential AP1 conducted along the small diameter nerve fiber S is at electrode E4, the small diameter nerve fiber S will be in the refractory period at the axial location adjacent electrode E4. The electrical pulse delivered by electrode E4 will, therefore, not evoke another action potential in the small diameter nerve fiber S at this axial location.

Additional action potentials AP5-AP8 may be similarly evoked in the large diameter nerve fiber L during respective times t=4x, t=5x, t=6x, and t=7x, while preventing additional action potentials from being evoked in the small diameter nerve fiber S. Once the last electrode E16 delivers the electrical pulse, the process can repeat starting with electrode E1 again. Although the initial electrical pulse is described as being delivered from electrode E1 at the end of the neurostimulation lead 12, it should be appreciated that the initial electrical pulse may be delivered from other electrodes. For example, the initial electrical pulse may be delivered from electrode E4, in which case, subsequent pulses can be sequentially delivered to electrodes E5-E16 at the conduction velocity of the small diameter nerve fiber. Furthermore, it is noted that although the action potentials have been described as being unidirectional in a specific direction (to the right in this case), the action potentials may be unidirectional in another direction (to the left) or bidirectional. If the action potentials are unidirectional to the right, the initial electrical pulse may be delivered from, e.g., electrode E16, and the subsequent electrical pulses can be sequentially delivered to electrodes E15, E14, and so forth, at the conduction velocity of the small diameter nerve fiber. If the action potentials are bidirectional, the initial electrical pulse may be delivered from, e.g., electrode E8, and the pairs of subsequent electrical pulses can be sequentially delivered from electrodes E7 and E9, then electrodes E6 and E10, electrodes E5 and E11, and so forth. Furthermore, although the stimulation pulses have been described as being sequentially delivered to immediately adjacent electrodes (i.e., electrode E1, then electrode E2, then electrode E3, etc.), the stimulation pulse may be sequentially delivered to electrodes that are not adjacent to each other (e.g., every other electrode, such as electrode E1, then electrode E3, then electrode E5, etc).

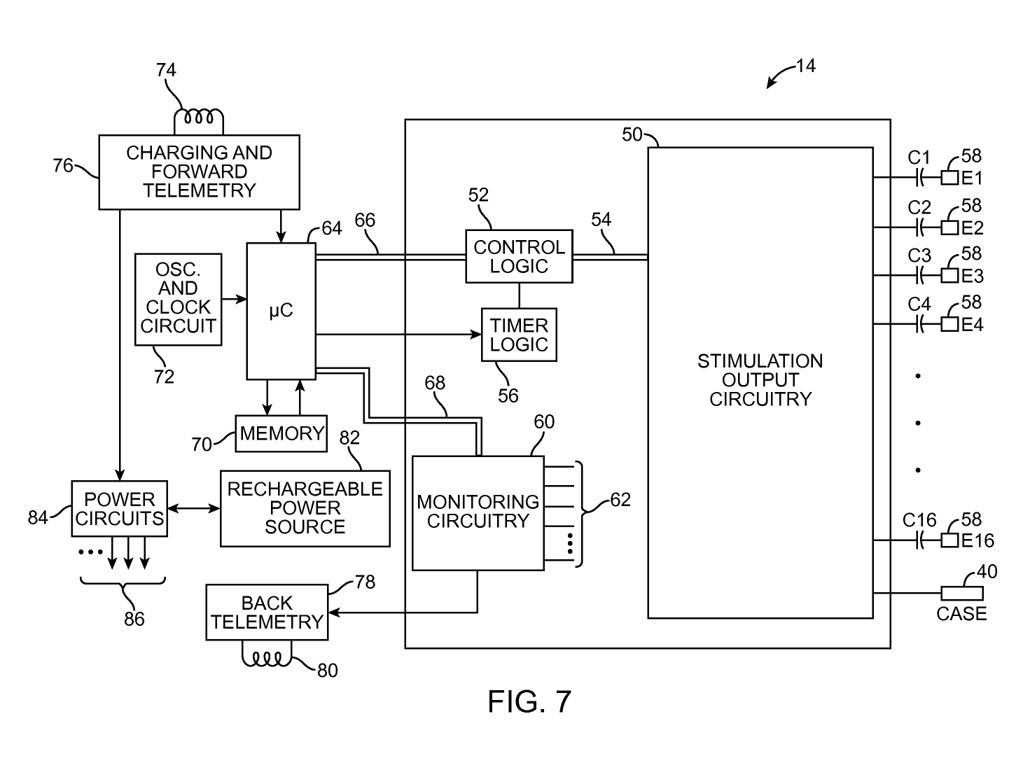

Turning next to FIG. 7, the main internal components of the IPG 14 will now be described. The IPG 14 includes stimulation output circuitry 50 configured for generating electrical stimulation energy in accordance with a defined pulsed waveform having a specified pulse amplitude, pulse rate, pulse width, pulse shape, and burst rate under control of control logic 52 over data bus 54. Control of the pulse rate and pulse width of the electrical waveform is facilitated by timer logic circuitry 56, which may have a suitable resolution, e.g., 10 μs. The stimulation energy generated by the stimulation output circuitry 50 is output via capacitors C1-C16 to electrical terminals 58 corresponding to the electrodes 26.

The analog output circuitry 50 may either comprise independently controlled current sources for providing stimulation pulses of a specified and known amperage to or from the electrodes 26, or independently controlled voltage sources for providing stimulation pulses of a specified and known voltage at the electrodes 26. The operation of this analog output circuitry, including alternative embodiments of suitable output circuitry for performing the same function of generating stimulation pulses of a prescribed amplitude and width, is described more fully in U.S. Pat. Nos. 6,516,227 and 6,993,384, which are expressly incorporated herein by reference.

The IPG 14 also comprises monitoring circuitry 60 for monitoring the status of various nodes or other points 62 throughout the IPG 14, e.g., power supply voltages, temperature, battery voltage, and the like. Notably, the electrodes 26 fit snugly within the tissue of the patient, and because the tissue is conductive, electrical measurements can be taken from the electrodes 26. The IPG 14 further comprises processing circuitry in the form of a microcontroller (μC) 64 that controls the control logic 52 over data bus 66, and obtains status data from the monitoring circuitry 60 via data bus 68. The IPG 14 additionally controls the timer logic 56. The IPG 14 further comprises memory 70 and oscillator and clock circuit 72 coupled to the microcontroller 64. The microcontroller 64, in combination with the memory 70 and oscillator and clock circuit 72, thus comprise a microprocessor system that carries out a program function in accordance with a suitable program stored in the memory 70. Alternatively, for some applications, the function provided by the microprocessor system may be carried out by a suitable state machine.

Thus, the microcontroller 64 generates the necessary control and status signals, which allow the microcontroller 64 to control the operation of the IPG 14 in accordance with a selected operating program and stimulation parameters. In controlling the operation of the IPG 14, the microcontroller 64 is able to individually generate stimulus pulses at the electrodes 26 using the analog output circuitry 50, in combination with the control logic 52 and timer logic 56, thereby allowing each electrode 26 to be paired or grouped with other electrodes 26, including the monopolar case electrode, to control the polarity, amplitude, rate, pulse width and channel through which the current stimulus pulses are provided. Significantly, the pulses generated at the electrodes 26 may be arranged in a pulse train, with each pulse being sequentially delivered to the electrodes 26 one-at-a-time.

The IPG 14 further comprises an alternating current (AC) receiving coil 74 for receiving programming data (e.g., the operating program and/or stimulation parameters) from the RC 16 and/or CP 18 in an appropriate modulated carrier signal, and charging and forward telemetry circuitry 76 for demodulating the carrier signal it receives through the AC receiving coil 74 to recover the programming data, which programming data is then stored within the memory 70, or within other memory elements (not shown) distributed throughout the IPG 14.

The IPG 14 further comprises back telemetry circuitry 78 and an alternating current (AC) transmission coil 80 for sending informational data sensed through the monitoring circuitry 60 to the RC 16 and/or CP 18. The back telemetry features of the IPG 14 also allow its status to be checked. For example, when the RC 16 and/or CP 18 initiates a programming session with the IPG 14, the capacity of the battery is telemetered, so that the RC 16 and/or CP 18 can calculate the estimated time to recharge. Any changes made to the stimulation parameters are confirmed through back telemetry, thereby assuring that such changes have been correctly received and implemented within the implant system. Moreover, upon interrogation by the RC 16 and/or CP 18, all programmable settings stored within the IPG 14 may be uploaded to the RC 16 and/or CP 18.

The IPG 14 further comprises a rechargeable power source 82 and power circuits 84 for providing the operating power to the IPG 14. The rechargeable power source 82 may, e.g., comprise a lithium-ion or lithium-ion polymer battery. The rechargeable battery 82 provides an unregulated voltage to the power circuits 84. The power circuits 84, in turn, generate the various voltages 86, some of which are regulated and some of which are not, as needed by the various circuits located within the IPG 14. The rechargeable power source 82 is recharged using rectified AC power (or DC power converted from AC power through other means, e.g., efficient AC-to-DC converter circuits, also known as “inverter circuits”) received by the AC receiving coil 74. To recharge the power source 82, an external charger (not shown), which generates the AC magnetic field, is placed against, or otherwise adjacent, to the patient's skin over the implanted IPG 14. The AC magnetic field emitted by the external charger induces AC currents in the AC receiving coil 74. The charging and forward telemetry circuitry 76 rectifies the AC current to produce DC current, which is used to charge the power source 82. While the AC receiving coil 74 is described as being used for both wirelessly receiving communications (e.g., programming and control data) and charging energy from the external device, it should be appreciated that the AC receiving coil 74 can be arranged as a dedicated charging coil, while another coil, such as coil 80, can be used for bi-directional telemetry.

Additional details concerning the above-described and other IPGs may be found in U.S. Pat. No. 6,516,227, U.S. Patent Publication No. 2003/0139781, and U.S. patent application Ser. No. 11/138,632, entitled “Low Power Loss Current Digital-to-Analog Converter Used in an Implantable Pulse Generator,” which are expressly incorporated herein by reference. It should be noted that rather than an IPG, the system 10 may alternatively utilize an implantable receiver-stimulator (not shown) connected to leads 12. In this case, the power source, e.g., a battery, for powering the implanted receiver, as well as control circuitry to command the receiver-stimulator, will be contained in an external controller inductively coupled to the receiver-stimulator via an electromagnetic link. Data/power signals are transcutaneously coupled from a cable-connected transmission coil placed over the implanted receiver-stimulator. The implanted receiver-stimulator receives the signal and generates the stimulation in accordance with the control signals.

Referring now to FIG. 8, one exemplary embodiment of an RC 16 will now be described. As previously discussed, the RC 16 is capable of communicating with the IPG 14, CP 18, or ETS 20. The RC 16 comprises a casing 100, which houses internal componentry (including a printed circuit board (PCB)), and a lighted display screen 102 and button pad 104 carried by the exterior of the casing 100. In the illustrated embodiment, the display screen 102 is a lighted flat panel display screen, and the button pad 104 comprises a membrane switch with metal domes positioned over a flex circuit, and a keypad connector connected directly to a PCB. In an optional embodiment, the display screen 102 has touchscreen capabilities. The button pad 104 includes a multitude of buttons 106, 108, 110, and 112, which allow the IPG 14 to be turned ON and OFF, provide for the adjustment or setting of modulation parameters within the IPG 14, and provide for selection between screens.

In the illustrated embodiment, the button 106 serves as an ON/OFF button that can be actuated to turn the IPG 14 ON and OFF. The button 108 serves as a select button that allows the RC 106 to switch between screen displays and/or parameters. The buttons 110 and 112 serve as up/down buttons that can be actuated to increase or decrease any of modulation parameters of the pulse generated by the IPG 14, including the pulse amplitude, pulse width, and pulse rate. For example, the selection button 108 can be actuated to place the RC 16 in a “Pulse Amplitude Adjustment Mode,” during which the pulse amplitude can be adjusted via the up/down buttons 110, 112, a “Pulse Width Adjustment Mode,” during which the pulse width can be adjusted via the up/down buttons 110, 112, and a “Pulse Rate Adjustment Mode,” during which the pulse rate can be adjusted via the up/down buttons 110, 112. Alternatively, dedicated up/down buttons can be provided for each stimulation parameter. Rather than using up/down buttons, any other type of actuator, such as a dial, slider bar, or keypad, can be used to increment or decrement the stimulation parameters. Significant to the present inventions, the selection button 108 can also be actuated to place the SCS system 10 in an “Large Fiber Stimulation” mode that utilizes the previous technique to maximize the activation of large diameter nerve fibers, while minimizing the activation of small diameter nerve fibers.

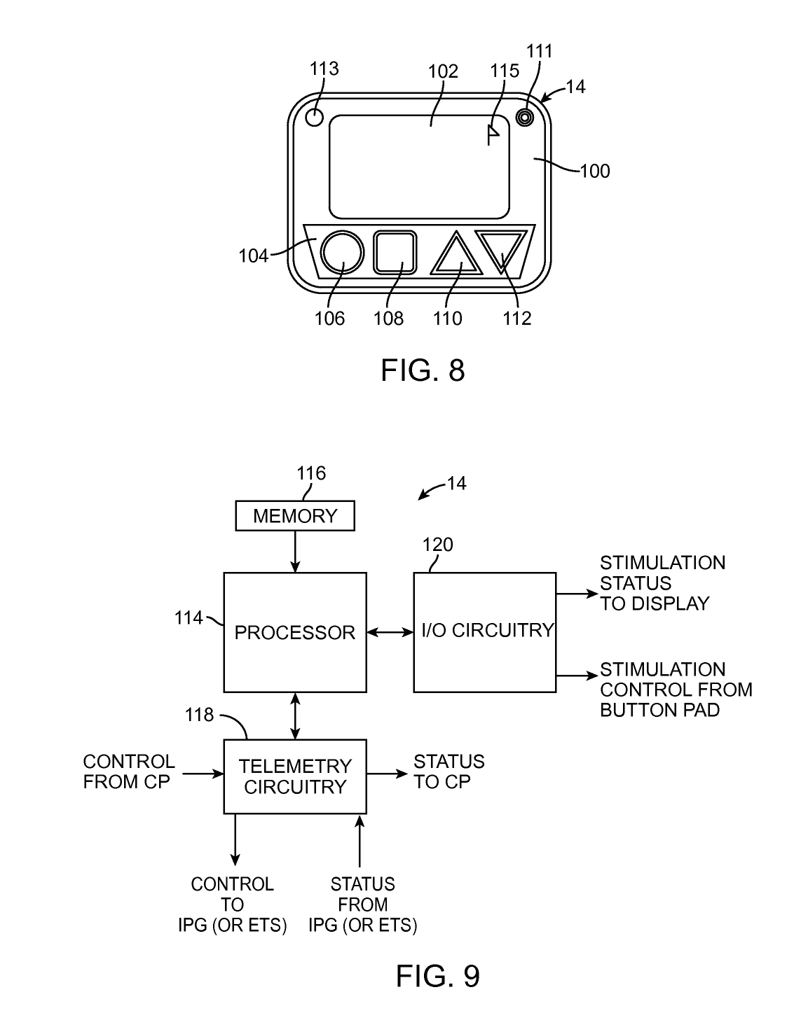

Referring to FIG. 9, the internal components of an exemplary RC 16 will now be described. The RC 16 generally includes a processor 114 (e.g., a microcontroller), memory 116 that stores an operating program for execution by the processor 114, as well as modulation parameters, input/output circuitry, and in particular, telemetry circuitry 118 for outputting modulation parameters to the IPG 14 and receiving status information from the IPG 14, and input/output circuitry 120 for receiving modulation control signals from the button pad 104 and transmitting status information to the display screen 102 (shown in FIG. 8). As well as controlling other functions of the RC 16, which will not be described herein for purposes of brevity, the processor 114 generates a plurality of modulation parameter sets that define the amplitude, phase duration, frequency, and waveform shape in response to the user operation of the button pad 104. These new modulation parameter sets would then be transmitted to the IPG 14 via the telemetry circuitry 118, thereby adjusting the modulation parameters stored in the IPG 14 and/or programming the IPG 14. The telemetry circuitry 118 can also be used to receive modulation parameters from the CP 18. Further details of the functionality and internal componentry of the RC 16 are disclosed in U.S. Pat. No. 6,895,280, which has previously been incorporated herein by reference.

Although the foregoing programming functions have been described as being at least partially implemented in the RC 16, it should be noted that these techniques may be at least, in part, be alternatively or additionally implemented in the CP 18.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. A neurostimulation system, comprising:
a plurality of electrical terminals configured for being respectively coupled to a plurality of electrodes;
analog output circuitry configured for generating a train of stimulation pulses; and
control circuitry configured for instructing the analog output circuitry to sequentially deliver a first one of the stimulation pulses to a first one of the electrical terminals, and a second one of the stimulation pulses to a second one of the electrical terminals.

2. The neurostimulation system of claim 1, further comprising a user interface configured for receiving an input from the user defining a pulse rate between the first and second stimulation pulses.

3. The neurostimulation system of claim 2, further comprising the plurality of electrodes.

4. The neurostimulation system of claim 3, wherein the plurality of electrodes is carried by a neurostimulation lead in an electrode column.

5. The neurostimulation system of claim 4, wherein the electrode column has an electrode spacing, and wherein the pulse rate is in the range
$$\frac{20 mm \text{/} ms}{x} \leq r \leq \frac{120 mm \text{/} ms}{x} ,$$
where r is the pulse rate in pulses per millisecond, and x is the electrode spacing in millimeters.

6. The neurostimulation system of claim 1, further comprising a housing containing the plurality of electrical terminals, the analog output circuitry, and the control circuitry.

7. A method of providing therapy to a patient with a plurality of electrodes extending along the longitudinal axes of a relatively large diameter nerve fiber and a relatively small diameter nerve fiber, comprising:
delivering electrical energy from a first one of the electrodes at an initial axial location, thereby evoking an initial action potential in the large diameter nerve fiber, and an initial action potential in the small diameter nerve fiber;
allowing the respective initial action potentials to be conducted along the large diameter nerve fiber and the small diameter nerve fiber in a first axial direction towards a second axial location; and
delivering electrical energy from a second one of the electrodes at the second axial location at an instant in time when the initial action potential conducted along the large diameter nerve fiber has passed the second axial location and the initial action potential conducted along the small diameter nerve fiber is at the second axial location, thereby allowing a subsequent action potential to be evoked in the large diameter nerve fiber while preventing a subsequent action potential from being evoked in the small diameter nerve fiber.

8. The method of claim 7, wherein the small diameter nerve fiber is undergoing a refractory period at the second axial location when the electrical energy is delivered from the second electrode at the second axial location.

9. The method of claim 7, wherein the large and small diameter nerve fibers are spinal cord nerve fibers.

10. The method of claim 7, wherein the spinal cord fibers are dorsal column nerve fibers.

11. The method of claim 7, wherein the series of electrodes are implanted within the patient.

12. The method of claim 7, wherein the series of electrodes are carried by a neurostimulation lead.

13. The method of claim 7, wherein the initial action potential in the large diameter nerve fiber has a conduction velocity greater than a conduction velocity of the initial action potential in the small diameter nerve fiber.

14. The method of claim 7, wherein the first and second electrodes are immediately adjacent each other.

15. The method of claim 7, wherein the therapy to the patient is provided using a train of pulses, the delivery of the electrical energy from the first and second electrodes respectively comprises sequentially delivering first and second pulses of the pulse train from the first and second electrodes.

16. The method of claim 7, wherein no electrical energy is delivered from the first electrode when the electrical energy is delivered from the second electrode.

17. The method of claim 7, further comprising:
allowing the respective initial action potentials to be conducted along the large diameter nerve fiber and the small diameter nerve fiber in the first axial direction from the second axial location towards a third axial location; and
delivering electrical energy from a third one of the electrodes at the third axial location at an instant in time when the initial action potential conducted along the large diameter nerve fiber has passed the third axial location and the initial action potential conducted along the small diameter nerve fiber is at the third axial location, thereby allowing another subsequent action potential to be evoked in the large diameter nerve fiber while preventing a subsequent action potential from being evoked in the small diameter nerve fiber.
